(19) United States
(12) Patent Application Publication
(10) Pub. No.: US 2026/0224230 A1
(43) Pub. Date: Aug. 6, 2026

(54) Length Restoring Lapidus Implant System

(71) Applicants: Natalia Reyes, San Antonio, TX (US); Thomas Zink, San Antonio, TX (US); Matthew Jackman, Sherman, TX (US); Joshua Nichols, Tallahassee, FL (US); Matthew Lining, Jasper, IN (US); Walter Strash, Canyon Lake, TX (US)

(72) Inventors: Natalia Reyes, San Antonio, TX (US); Thomas Zink, San Antonio, TX (US); Matthew Jackman, Sherman, TX (US); Joshua Nichols, Tallahassee, FL (US); Matthew Lining, Jasper, IN (US); Walter Strash, Canyon Lake, TX (US)

(21) Appl. No.: 19/043,851

(22) Filed: Feb. 3, 2025

(51) Int. Cl. A61B 17/17; A61B 17/56; A61F 2/30; A61F 2/42

(52) U.S. Cl. CPC A61B 17/1775; A61F 2/30734; A61F 2/4225; A61B 2017/565; A61F 2002/30736

(57) ABSTRACT

A length restoring Lapidus implant system is disclosed. The system is designed for a first metatarsal-cuneiform lengthening arthrodesis to correct hallux valgus deformities. The system comprises an anatomically shaped PEEK (polyether ether ketone) wedge implant used to maintain length and optimize positioning of the first ray in both transverse and sagittal planes and instrumentation that allows for rotational correction, as well as providing compression.

### FIELD OF THE INVENTION

The present invention relates generally to a medical device for use in the field of podiatry. More particularly, the present invention relates to an implant system indicated for a first metatarsal-cuneiform lengthening arthrodesis to correct hallux valgus deformities. The disclosed system allows for tri-planar correction of the first ray. Accordingly, the present disclosure makes specific reference thereto. Nonetheless, it is to be appreciated that aspects of the present invention are also equally applicable to other like applications, devices and methods of manufacture.

### BACKGROUND

The Lapidus procedure is commonly used to correct a hallux valgus (bunion) deformity, which is a lateral deviation of the great toe, with subsequent hypermobility (or laxity). The deformity often causes abnormal fitting of shoes and causes irritation of the patient's skin in places where the foot rubs against the footwear, which often leads to inflammation and pain. A bunion is caused by a malalignment of the first metatarsal and the proximal phalanx of the hallux. The formation of a bunion alters the distribution of ground reactive forces throughout the human gait cycle, which can also lead to irritation, calluses, blistering and ulceration of the skin, in addition to making walking difficult.

The Lapidus procedure is also commonly used to repair failed surgeries. Typically, a wedge of bone is removed in a biplanar direction at the distal end of the cuneiform, which will provide correction of the deformity and typically results in shortening of the great toe. The result of this shortening is a shift in weight distribution to the second ray, which can result in metatarsalgia. When the first ray is shortened the function of the patient's sesamoids may also be affected because of the change in weight distribution on the sesamoids. Currently to correct the shortening of the great toe when doing a Lapidus procedure, the accepted practice is for surgeons to make a straight transverse cut on the metatarsal, then cut a wedge out of the cuneiform to obtain realignment of the intermetatarsal angle as determined by the surgeon and insert a block of bone into the joint. The block of bone is then shaped by the surgeon until it fits within the joint. However, the shape of bone fails to help correct the angle. Further, blood supply to this joint can be limited in certain patients and using the overly processed bone makes it difficult to incorporate and heal which makes the bone prone to failure. It is well known that blood supply consideration to the joint and anatomical height and weight bearing through the joint are all concerns for healing the Lapidus procedure.

Additionally, the metatarsal-phalangeal joint, when fused, is commonly denuded of cartilage by either using cup and cone reamers to minimize a loss of length and to provide versatility in final positioning or by making transverse type cuts using a saw blade. Generally, the cartilage surfaces of the metatarsal and proximal phalanx are removed, and the end of the proximal phalanx is aligned with the end of the metatarsal with the two bones being fused together using screws, wires, or plates. In the case of revision surgeries of the metatarsal-phalangeal joint, the first ray may be shortened by 5-10 mm.

Accordingly, in view of the deficiencies of the current implants and methods of performing the Lapidus procedure and fusion of the metatarsal-phalangeal joint, it would be desirable to develop devices, instrumentation, and methods which allow lengthening arthrodesis to correct hallux valgus deformities. Specifically, devices, instrumentation and methods which utilize an anatomically shaped implant used to maintain length and optimize positioning of the first ray in both transverse and sagittal planes.

The subject matter disclosed and claimed herein, in one embodiment thereof, comprises a length restoring Lapidus implant system. The system is designed for a first metatarsal-cuneiform lengthening arthrodesis to correct hallux valgus deformities. The system comprises an anatomically shaped PEEK (polyether ether ketone) wedge implant used to maintain length and optimize positioning of the first ray in both transverse and sagittal planes and instrumentation that allows for rotational correction, as well as providing compression. The wedge implant is provided in various length maintaining heights and angle variations for correction of the first ray in both transverse and sagittal planes. The instrumentation includes at least a rotational jig, a parallel cut guide, and an interfragmentary screw sleeve, among other instruments. The rotational jig allows for rotational correction to correct sesamoid positioning. When the rotational jig is combined with the implant, the system results in tri-planar correction. The parallel cut guide allows the surgeon to resect as much bone and cartilage as desired. Using the designated cut guide slots, the exact amount of length removed will be replaced with the corresponding wedge implant height to maintain the natural length of the first ray. The interfragmentary screw sleeve is used to target the space between the wedge screws and provide compression through the wedge implant and between the first metatarsal and medial cuneiform.

In this manner, the length restoring Lapidus implant system of the present invention accomplishes all of the forgoing objectives and provides users with a system that allows the surgeon to maintain the length of the first ray. The wedge implant of the system provides multiple height and angle variations to optimize positioning of the first ray. The instrumentation of the system allows for correction of the axial sesamoids while correcting intermetatarsal angles.

### SUMMARY OF THE INVENTION

The following presents a simplified summary in order to provide a basic understanding of some aspects of the disclosed innovation. This summary is not an extensive overview, and it is not intended to identify key or critical elements or to delineate the scope thereof. Its sole purpose is to present some concepts in a simplified form as a prelude to the more detailed description that is presented later.

The subject matter disclosed and claimed herein, in one embodiment thereof, comprises a length restoring Lapidus implant system. The system is designed for a first metatarsal-cuneiform lengthening arthrodesis to correct hallux valgus deformities. The system comprises an anatomically shaped PEEK (polyether ether ketone) wedge implant used to maintain length and optimize positioning of the first ray in both transverse and sagittal planes and instrumentation that allows for rotational correction, as well as providing compression.

The wedge implant is provided in various length maintaining heights and angle variations for correction of the first ray in both transverse and sagittal planes. The wedge implant is configured from a known biocompatible implant material and is used for aiding or providing internal fixation of calcaneus, tarsal, and metatarsal bones of the foot in cases such as, but not limited to, filling an osteotomy or expanding and correcting an angular deformity in the foot.

In one embodiment, the wedge implant comprises a generally wedge-shaped body, with the body having a generally rounded top which recedes in to a bottom with a smaller radius, a generally planar first side, and a generally planar second side. The first and second sides each slope inwardly from the top to the bottom. However, the dimensions and angles of the wedge implant may be changed as desired in order to accommodate varying anatomies, purposes, and desired outcomes. It should also be appreciated that the wedge implant and/or its principles are applicable to other bones of the body such as, but not limited to, the bones of the hand.

Further, the body of the wedge implant also comprises two bores each of which are dimensioned to receive a bone screw, such that the head of the bone screw is held by the body of the wedge implant and a portion of the tip and/or shank of the bone screw extends from the body for receipt in a foot bone. The first bone screw bore extends between the top and the first side such that the tip of a bone screw extends from the first side at an angle thereof. The opening of the first bone screw bore on the top of the body is situated proximate to and on the edge between the top and the second side creating the angled bone screw bore. The second bone screw bore extends between the top and the second side such that the tip of a bone screw extends from the second side at an angle thereof. The opening of the second bone screw bore on the top of the body is situated proximate to and on the edge between the top and the first side creating the angled second bone screw bore. Both bone screw bore openings can be designed for different angulation. Further, both bone screw bore openings on the top of the body are sized and configured to capture and retain the head of a bone screw. Moreover, both bone screw bore openings on the top are configured such that the heads of the bone screws are countersunk relative to the surface of the top.

In another embodiment, the wedge implant body comprises an opening that extends from one side to the second side. The opening can be any suitable shape and size as is known in the art, depending on the needs and/or wants of a user. The opening is used to receive and hold bone graft material. As such, the opening may be considered a bone graft window or simply a graft window. The bone graft material may be of any suitable kind as is known in the art. Moreover, the graft window may comprise two or more openings that each extend from one side to the second side. These graft openings can be shaped as desired. In use, the wedge implant body can be placed in a graft packing block, and then filled with graft material.

In addition to the bone graft window, the body comprises features for aiding in and/or promoting fusion between two foot bones or foot bone portions. These features also aid in keeping the body in place. Particularly, a portion of the one side has teeth, serrations or any other suitable anti-migration features, while a portion of the other side likewise has teeth, serrations or any other suitable anti-migration features. The teeth surround the graft window or at least are present about the top and sides of the graft window. Typically, the upper portions of the sides proximate to the top portions of the sides, preferably, but not necessarily, lack teeth, serrations, or other suitable anti-migration features.

In another embodiment, the body of the wedge implant comprises a threaded bore/insertion feature that is provided for engagement with an insertion instrument and/or drill guide, or other insertion instruments as is known in the art.

In yet another embodiment, the wedge implant body includes a through hole, slot or the like in the distal aspect of the implant body. This allows for the insertion of a metallic component, such as titanium or tantalum to allow for the visibility of the extent of the implant body in radiography, x-ray, or the like.

In another embodiment, the wedge implant is manufactured as an additively printed titanium or PEEK component. Nonetheless, the wedge implant can also be manufactured as a machined titanium alloy, stainless steel, or any other suitable biocompatible material component as is known in the art. Further, the wedge implant can include an inner mesh structure, which is generated by 3D printed PEEK, titanium, or any other suitable biocompatible material as is known in the art, and which acts as a scaffold for bone.

Additionally, the instrumentation of the system includes at least a rotational jig, a parallel cut guide, and an interfragmentary screw sleeve, among other instruments. Other suitable instruments as is known in the art can be utilized to accommodate varying anatomies, purposes, and desired outcomes.

Specifically, the rotational jig allows for rotational correction to correct sesamoid positioning. When the rotational jig is combined with the wedge implant, the system results in tri-planar correction. The rotational jig comprises a base component with extended opposing ends. The opposing ends extend generally perpendicular from the base component, with each end comprising at least one through hole, bore or other suitable opening as is known in the art, at an end farthest away from the base component. The through hole is sized to receive a pin or other suitable securing means as is known in the art, depending on the needs and/or wants of a user. Typically, the rotational jig is attached with a Steinmann pin or other suitable securing means as is known in the art, to the medial cuneiform or navicular. Further, the middle of the base component comprises a slotted step arch comprising a plurality of slotted steps, which is in communication with a geared rack and pinion system. The geared rack and pinion system further comprises a control knob, that when turned moves the slotted step arch distally or proximally. Another pin is placed through the slotted step arch of the rotational jig into the medial aspect of the first metatarsal. The pin is used as a joystick, gliding up the step arch into the next highest slot to perform coronal correction (i.e., frontal plane correction) and rotate the sesamoids plantar. The geared rack and pinion system is then used to compress and distract as needed.

Furthermore, the parallel cut guide allows the surgeon to resect as much bone and cartilage as desired. Using the designated cut guide slots, the exact amount of length removed will be replaced with the corresponding wedge implant height to maintain the natural length of the first ray. The parallel cut guide comprises a curved base component with a paddle for insertion into the joint space, positioned at a rear of the base component. The cut guide also comprises a plurality of parallel cut guides/slots positioned on the base component surface. The base component further comprises a plurality of through holes for fixation options and a threaded through hole for the joystick positioned as extensions of the base component. In use, the paddle of the cut guide is placed into the first tarsometatarsal (TMT) joint. The cut guide joystick is then threaded into the cut guide and used to position the cut guide more dorsally or medially. K-wires can then be placed in any of the through holes in the cut guide to fixate the guide in its optimal position. A sagittal saw (not shown) is then used to make equal, parallel cuts on the medial cuneiform and first metatarsal. The slots in the guide correlate to the various implant heights. Cut guide and remaining bone slivers are removed. The geared rack and pinion system of the rotational jig, attached to the slotted arch and pinned to the first metatarsal, is used to distract the joint space.

Additionally, an intermetatarsal (IM) angle reducer can be utilized to compress the IM angle. The IM angle reducer comprises a C-shaped clamp and screw component, wherein an end of the clamp comprises a second metatarsal hook which engages the second metatarsal and an end of the screw comprises a first metatarsal hook which engages the first metatarsal to compress the IM angle. Specifically, the second metatarsal hook hooks around the lateral aspect of the second metatarsal and the first metatarsal hook is placed around the medial aspect of the first metatarsal. The knob of the screw is then threaded clockwise to compress the IM angle around a trial device. The rotational jig can then be used to further compress the joint around the trial device to visualize correction. The optimally sized implant then replaces the trial device in the joint space. The rotational jig is then used to compress the first metatarsal to the implant.

Further, a wedge screw sleeve is utilized to insert the temporary fixation screws. The wedge screw sleeve comprises a shaft with corresponding angled drill guide towers. The shaft of the wedge screw sleeve is cannulated, allowing the wedge screw sleeve to slide over an inserter device. Specifically, the wedge screw sleeve is slid over the inserter and the drill guides are aligned with the insertion features on the wedge implant. Once in place, the wedge screw sleeve is slid over the inserter and aligned with the insertion features of the wedge. The attached knob is threaded clockwise to engage the inserter shaft and lock the sleeve in its fully seated position within the wedge's insertion feature. Drilling and inserting of the temporary fixation screws can then be done through the drill guide towers attached to the wedge screw sleeve. Furthermore, the wedge screw sleeve can also act as a depth gauge for the screws.

Finally, the interfragmentary screw sleeve is used to target the space between the wedge screws and provide compression through the wedge implant and between the first metatarsal and medial cuneiform. The interfragmentary screw sleeve comprises a shaft with corresponding angled drill guide towers. The drill guide towers can be positioned at any suitable angle as is known in the art, as long as the interfragmentary screws do not contact the wedge screws. The shaft of the interfragmentary screw sleeve is cannulated, allowing the interfragmentary screw sleeve to slide over the inserter device. Specifically, the knob from the wedge screw sleeve is un-thread and removed, and the wedge screw sleeve is then removed, while keeping the inserter threaded into the wedge implant. The interfragmentary screw sleeve is then slid over the inserter and the drill guides are positioned. Once in place, a threaded knob is slid over the inserter shaft to lock the interfragmentary screw sleeve to the shaft and ensure the sleeve is fully seated in the insertion feature of the wedge implant. K-wires are then utilized to ensure the correct trajectory of the two opposing interfragmentary screws. Once the interfragmentary screws are placed, the interfragmentary screw sleeve and inserter are removed, and the patient is closed.

Numerous benefits and advantages of this invention will become apparent to those skilled in the art to which it pertains, upon reading and understanding the following detailed specification.

To the accomplishment of the foregoing and related ends, certain illustrative aspects of the disclosed innovation are described herein in connection with the following description and the annexed drawings. These aspects are indicative, however, of but a few of the various ways in which the principles disclosed herein can be employed and are intended to include all such aspects and their equivalents. Other advantages and novel features will become apparent from the following detailed description when considered in conjunction with the drawings.

### BRIEF DESCRIPTION OF THE DRAWINGS

The description refers to provided drawings in which similar reference characters refer to similar parts throughout the different views, and in which:

FIGS. 1A-1D illustrate multiple views of the wedge implant of the length restoring Lapidus implant system of the present invention in accordance with the disclosed architecture;

FIGS. 2A-2B illustrate multiple views of the rotational jig of the length restoring Lapidus implant system of the present invention in accordance with the disclosed architecture;

FIGS. 3A-3D illustrate multiple views of the cut guide of the length restoring Lapidus implant system of the present invention in accordance with the disclosed architecture;

FIGS. 4A-4B illustrate multiple views of the IM angle reducer of the length restoring Lapidus implant system of the present invention in accordance with the disclosed architecture;

FIG. 5 illustrates a perspective view of the trial wedge implant and inserter of the length restoring Lapidus implant system of the present invention in accordance with the disclosed architecture;

FIGS. 6A-6B illustrate multiple views of the graft packing block of the length restoring Lapidus implant system of the present invention in accordance with the disclosed architecture;

FIGS. 7A-7D illustrate multiple views of the wedge screw sleeve of the length restoring Lapidus implant system of the present invention in accordance with the disclosed architecture;

FIGS. 8A-8B illustrate multiple views of the interfragmentary screw sleeve of the length restoring Lapidus implant system of the present invention in accordance with the disclosed architecture; and

FIG. 9 illustrates a perspective view of the final configuration of the length restoring Lapidus implant system of the present invention in accordance with the disclosed architecture.

### DETAILED DESCRIPTION

The innovation is now described with reference to the drawings, wherein like reference numerals are used to refer to like elements throughout. In the following description, for purposes of explanation, numerous specific details are set forth in order to provide a thorough understanding thereof. It may be evident, however, that the innovation can be practiced without these specific details. In other instances, well-known structures and devices are shown in block diagram form in order to facilitate a description thereof. Various embodiments are discussed hereinafter. It should be noted that the figures are described only to facilitate the description of the embodiments. They are not intended as an exhaustive description of the invention and do not limit the scope of the invention. Additionally, an illustrated embodiment need not have all the aspects or advantages shown. Thus, in other embodiments, any of the features described herein from different embodiments may be combined.

In this application, the words proximal, distal, anterior or plantar, posterior or dorsal, medial and lateral are defined by their standard usage for indicating a particular part or portion of a bone, or directional terms of reference, according to the relative disposition of the natural bone. For example, “proximal” means the portion of a bone nearest the torso, while “distal” indicates the portion of the bone farthest from the torso. As an example of directional usage of the terms, “anterior” refers to a direction towards the front side of the body, “posterior” refers to a direction towards the back side of the body, “medial” refers to a direction towards the midline of the body and “lateral” refers to a direction towards the sides or away from the midline of the body. Further, specifically in regard to the foot, the term “dorsal” refers to the top of the foot and the term “plantar” refers the bottom of the foot.

Similarly, positions or directions may be used herein with reference to anatomical structures or surfaces. For example, as the current devices, instrumentation and methods are described herein with reference to use with the bones of the foot, the bones of the foot, ankle and lower leg may be used to describe the surfaces, positions, directions or orientations of the devices, instrumentation and methods. Further, the devices, instrumentation and methods, and the aspects, components, features and the like thereof, disclosed herein are described with respect to one side of the body for brevity purposes. However, as the human body is relatively symmetrical or mirrored about a line of symmetry (midline), it is hereby expressly contemplated that the devices, instrumentation and methods, and the aspects, components, features and the like thereof, described and/or illustrated herein may be changed, varied, modified, reconfigured or otherwise altered for use or association with another side of the body for a same or similar purpose without departing from the spirit and scope of the invention. For example, the devices, instrumentation and methods, and the aspects, components, features and the like thereof, described herein with respect to the right foot may be mirrored so that they likewise function with the left foot. Further, the devices, instrumentation and methods, and the aspects, components, features and the like thereof, disclosed herein are described with respect to the foot for brevity purposes, but it should be understood that the devices, instrumentation, and methods may be used with other bones of the body having similar structures, for example the upper extremity, and more specifically, with the bones of the wrist, hand, and arm.

As noted above, there is a long felt need in the art for devices, instrumentation, and methods which allow lengthening arthrodesis to correct hallux valgus deformities. Specifically, devices, instrumentation and methods which utilize an anatomically shaped implant used to maintain length and optimize positioning of the first ray in both transverse and sagittal planes.

The present invention, in one exemplary embodiment, is a novel length restoring Lapidus implant system. The system comprises an anatomically shaped PEEK (polyether ether ketone) wedge implant used to maintain length and optimize positioning of the first ray in both transverse and sagittal planes and instrumentation that allows for rotational correction, as well as providing compression. The wedge implant is provided in various length maintaining thicknesses and angle variations for correction of the first ray in both transverse and sagittal planes. The instrumentation includes at least a rotational jig, a parallel cut guide, and an interfragmentary screw sleeve, among other instruments.

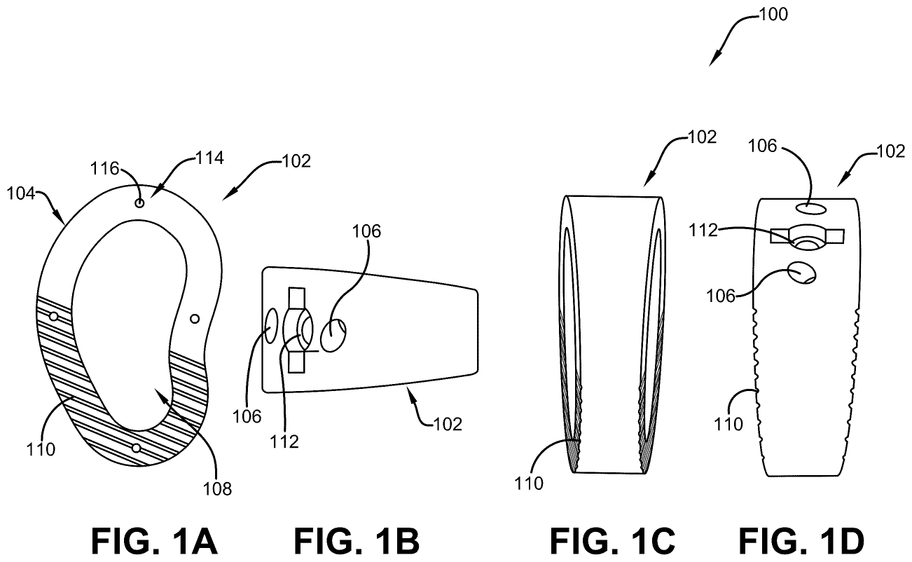

Referring initially to the drawings, FIGS. 1-9 illustrate multiple views of the length restoring Lapidus implant system 100 of the present invention. In the present embodiment, the length restoring Lapidus implant system 100 is an improved Lapidus system that maintains length and optimizes positioning of the first ray in both transverse and sagittal planes. The system 100 is especially designed to allow any doctor, surgeon, etc., or any other suitable user as is known in the art, to maintain the length of and optimize positioning of the first ray and allow for correction of the axial sesamoids. More specifically, the system 100 comprises a wedge implant 102 used to maintain length and optimize positioning of the first ray in both transverse and sagittal planes and instrumentation 200, 300, 400, 700, 800 that allows for rotational correction, as well as providing compression.

Specifically, as illustrated in FIGS. 1A-D, one embodiment of the wedge implant 102 is shown in a distal view, a dorsal view, a lateral view, and a medial view, respectively. The wedge implant 102 is made to a desired restoration length necessary for the user receiving the implant 102 and has an outer anatomical profile that mimics the anatomical considerations of the bones it is being inserted between. For example, as depicted in FIGS. 1A-D, the outer anatomical profile of the wedge implant 102 on the proximal and distal sides is cut to mimic the shapes of the medial cuneiform and metatarsal bones, which is an oblong shape, or more preferably is a kidney shape. In addition, the wedge implant 102 is cut with a desired angle thereby creating a desired angular offset between the medial cuneiform and the metatarsal. For example, in the Lapidus wedge implant 102 the desired angulation may be used to correct valgus and plantar angulation of the bones by tapering the wedge implant 102 from the medial side to the lateral side and from the dorsal side to the plantar side. Furthermore, the wedge implant 102 may have heights ranging from about 20 mm to 40 mm, widths ranging from about 10 mm to 30 mm, and thicknesses ranging from about 0 mm to 20 mm at the dorsal side and the medial side and tapering from the dorsal side to plantar side and medial side to lateral side at an angle ranging from about 0 degrees to 20 degrees. More preferably, the wedge implants 102 have a height of about 24 mm, a width of about 21 mm at the dorsal medial corner, and functional thickness of 5 mm and 5 degrees, 8 mm and 8 degrees, 10 mm and 10 degrees, 12 mm and 12 degrees, and 14 mm and 0 degrees. It is also contemplated that the taper of the thickness of the wedge implant 102 may be from the dorsal-medial corner to the plantar-lateral corner, or any other suitable direction as is known in the art. However, the dimensions and angles of the wedge implant 102 may be changed as desired in order to accommodate varying anatomies, purposes, and desired outcomes. It should also be appreciated that the wedge implant 102 and/or its principles are applicable to other bones of the body such as, but not limited to, the bones of the hand.

Further, the wedge implant 102 comprises a body 104 comprising at least two bores 106 each of which are dimensioned to receive a bone screw (not shown), such that the head of the bone screw is held by the body 104 of the wedge implant 102 and a portion of the tip and/or shank of the bone screw extends from the body 104 for receipt in a foot bone. Any suitable number of bores 106 can be utilized as is known in the art, depending on the needs and/or wants of a user. The first bone screw bore 106 extends between the top and the first side, such that the tip of a bone screw extends from the first side at an angle thereof. The opening of the first bone screw bore 106 on the top of the body 104 is situated proximate to and on the edge between the top and the second side creating the angled bone screw bore 106. The second bone screw bore 106 extends between the top and the second side such that the tip of a bone screw extends from the second side at an angle thereof. The opening of the second bone screw 106 bore on the top of the body 104 is situated proximate to and on the edge between the top and the first side creating the angled second bone screw bore 106. Both bone screw bore openings 106 can be designed for different angulation. Further, both bone screw bore openings 106 on the top of the body 104 are sized and configured to capture and retain the head of a bone screw. Moreover, both bone screw bore openings 106 on the top are configured such that the heads of the bone screws are countersunk relative to the surface of the top.

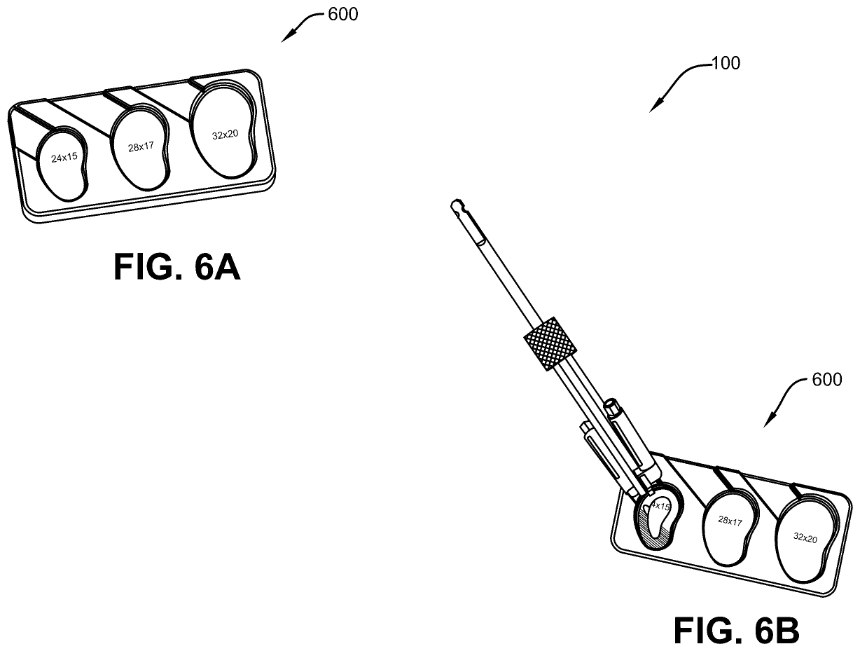

Additionally, the wedge implant body 104 comprises an opening 108 that extends from one side to the second side. The opening 108 can be any suitable shape and size as is known in the art, depending on the needs and/or wants of a user. The opening 108 is used to receive and hold bone graft material (not shown). As such, the opening 108 may be considered a bone graft window or simply a graft window. The bone graft material may be of any suitable kind as is known in the art. Moreover, the graft window 108 may comprise two or more openings that each extend from one side to the second side. These graft openings 108 can be shaped as desired. In use, the wedge implant body 104 can be placed in a graft packing block, and then filled with graft material (as shown in FIG. 6).

In addition to the bone graft window 108, the body 104 comprises features 110 for aiding in and/or promoting fusion between two foot bones or foot bone portions. These features 110 also aid in keeping the body 104 in place. Particularly, a portion of the one side has teeth, serrations or any other suitable anti-migration features 110, while a portion of the other side likewise has teeth, serrations or any other suitable anti-migration features 110. The teeth 110 surround the graft window 108 or at least are present about the top and sides of the graft window 108. Typically, the upper portions of the sides proximate to the top portions of the sides, preferably, but not necessarily, lack teeth, serrations, or other suitable anti-migration features 110.

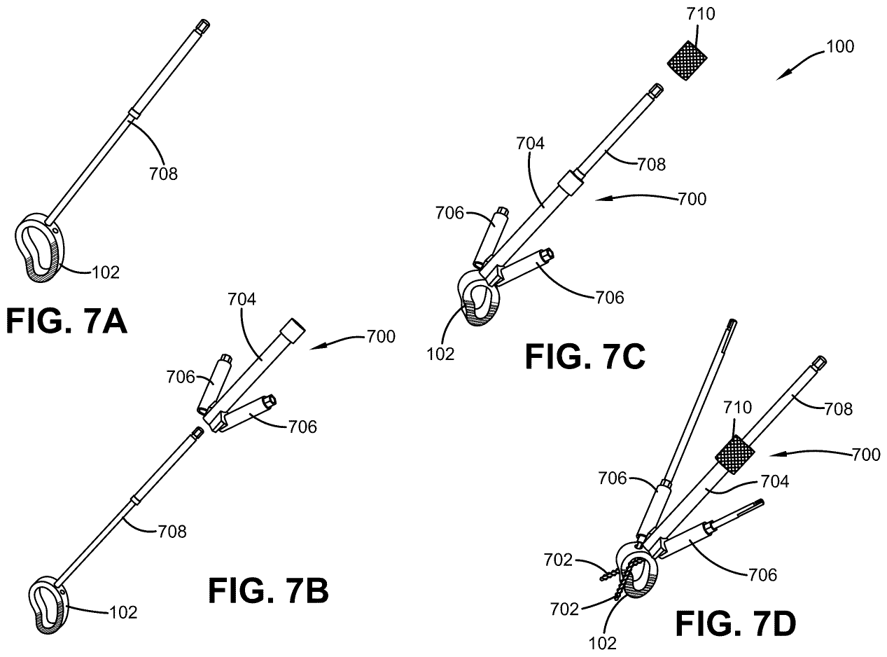

Further, the body 104 of the wedge implant 102 comprises a threaded bore/insertion feature 112 that is provided for engagement with an insertion instrument (as shown in FIG. 7) and/or drill guide, or other insertion instruments as is known in the art. In another embodiment, the wedge implant body 104 includes a through hole, slot 114 or the like in the distal aspect of the implant body 104. This allows for the insertion of a metallic component 116, such as titanium or tantalum to allow for the visibility of the extent of the implant body 104 in radiography, x-ray, or the like.

In another embodiment, the wedge implant 102 is manufactured as an additively printed titanium or PEEK component. Specifically, the wedge implant 102 is additively printed and able to be manufactured in a variety of sizes as well as to be customizable to fit the exact specifications/measurements of the particular user. Nonetheless, the wedge implant 102 can also be manufactured as a machined titanium alloy, stainless steel, or any other suitable biocompatible material component as is known in the art. Further, the wedge implant 102 can include an inner mesh structure (not shown), or any other suitable structure as is known in the art, which is generated by 3D printed PEEK, titanium, or any other suitable biocompatible material as is known in the art, and which acts as a scaffold for bone.

Additionally, the instrumentation of the system 100 includes at least a rotational jig 200, a parallel cut guide 300, and an interfragmentary screw sleeve 800, among other instruments. Other suitable instruments as is known in the art can be utilized to accommodate varying anatomies, purposes, and desired outcomes.

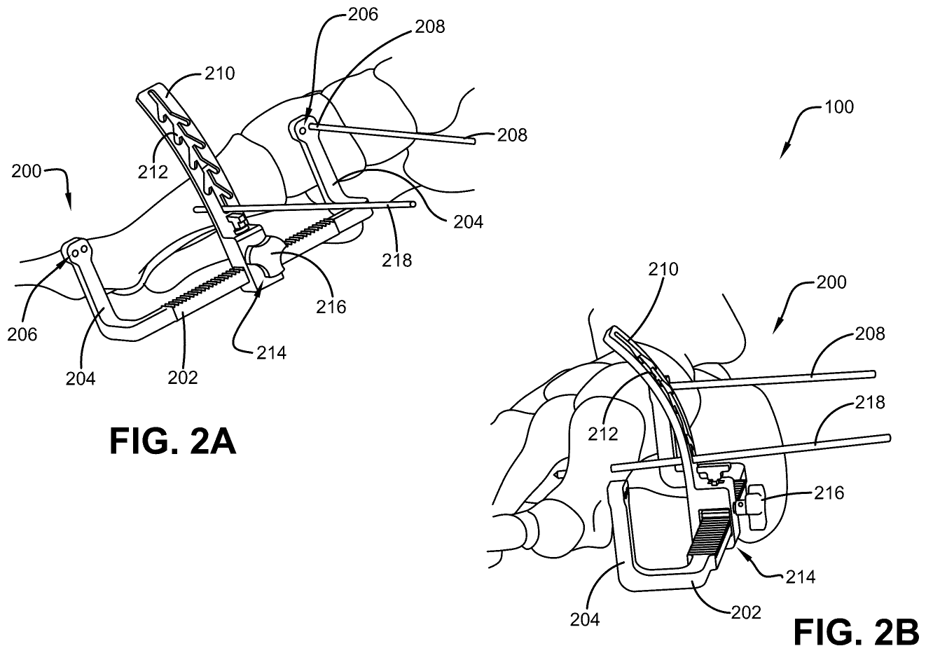

FIGS. 2A-B illustrate multiple views of the rotational jig 200 which allow for rotational correction to correct sesamoid positioning. When the rotational jig 200 is combined with the wedge implant 102, the system 100 results in tri-planar correction. The rotational jig 200 comprises a base component 202 with extended opposing ends 204. The opposing ends 204 extend generally perpendicular from the base component 202, with each end 204 comprising at least one through hole, bore or other suitable opening 206 as is known in the art, at an end farthest away from the base component 202. The through hole 206 is sized to receive a pin 208 or other suitable securing means as is known in the art, depending on the needs and/or wants of a user. Typically, the rotational jig 200 is attached with a Steinmann pin 208 or other suitable securing means as is known in the art, to the medial cuneiform or navicular. Further, the middle of the base component 202 comprises a slotted step arch 210 comprising a plurality of slotted steps 212, which is in communication with a geared rack and pinion system 214. The geared rack and pinion system 214 further comprises a control knob 216, that when turned moves the slotted step arch 210 distally or proximally. Another pin 218 is placed through the slotted step arch 210 of the rotational jig 200 into the medial aspect of the first metatarsal. The pin 218 is used as a joystick, gliding up the step arch 210 into the next highest slot 212 to perform coronal correction (i.e., frontal plane correction) and rotate the sesamoids plantar. The geared rack and pinion system 214 is then used to compress and distract as needed.

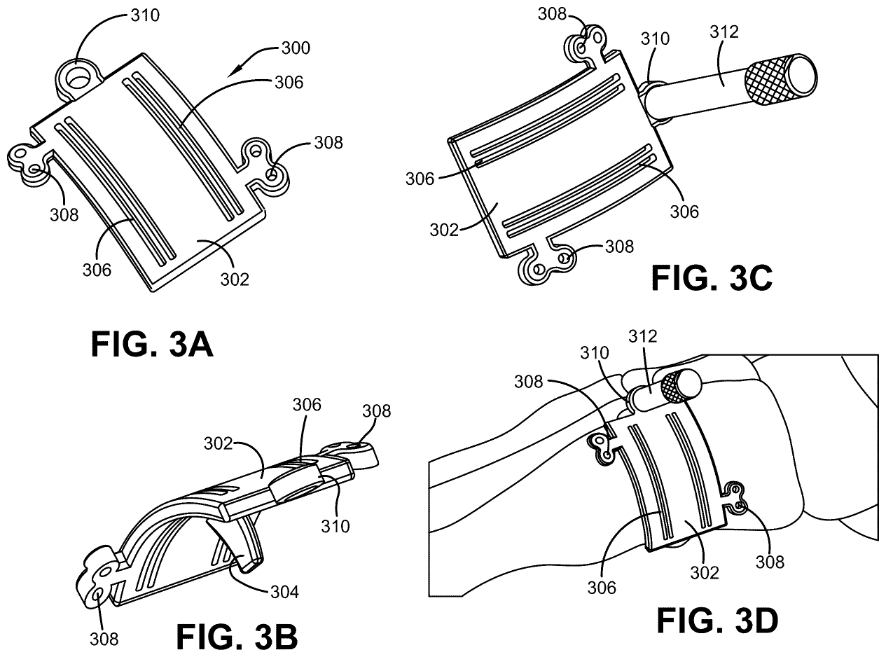

FIGS. 3A-D illustrate multiple views of the parallel cut guide 300 which allows the surgeon to resect as much bone and cartilage as desired. Using the designated cut guide slots, the exact amount of length removed will be replaced with the corresponding wedge implant 102 thickness to maintain the natural length of the first ray. The parallel cut guide 300 comprises a curved base component 302 with a paddle 304 for insertion into the joint space, positioned at a rear of the base component 302. The cut guide 300 also comprises a plurality of parallel cut guides/slots 306 positioned on the base component 302 surface. The base component 302 further comprises a plurality of through holes 308 for fixation options and a threaded through hole 310 for the joystick 312 positioned as extensions of the base component 302. In use, the paddle 304 of the cut guide 300 is placed into the first tarsometatarsal (TMT) joint. The cut guide joystick 312 is then threaded into the threaded hole 310 and used to position the cut guide 300 more dorsally or medially. K-wires (not shown) can then be placed in any of the through holes 308 in the cut guide 300 to fixate the guide 300 in its optimal position. A sagittal saw (not shown) is then used to make equal, parallel cuts on the medial cuneiform and first metatarsal. The slots 306 in the guide 300 correlate to the various implant heights. Cut guide 300 and remaining bone slivers are removed. The geared rack and pinion system 214 of the rotational jig 200, attached to the slotted arch 210 and pinned to the first metatarsal, is used to distract the joint space.

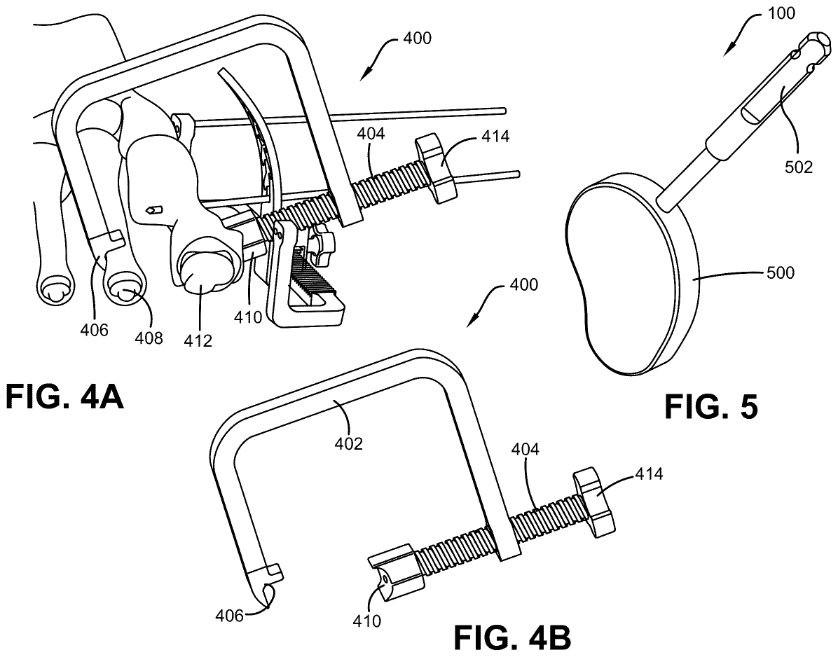

FIGS. 4A-B illustrate multiple views of the intermetatarsal (IM) angle reducer 400 which can be utilized to compress the IM angle. The IM angle reducer 400 comprises a C-shaped clamp 402 and screw component 404, wherein an end of the clamp 402 comprises a second metatarsal hook 406 which engages the second metatarsal 408 and an end of the screw 404 comprises a first metatarsal hook 410 which engages the first metatarsal 412 to compress the IM angle. Specifically, the second metatarsal hook 406 hooks around the lateral aspect of the second metatarsal 408 and the first metatarsal hook 410 is placed around the medial aspect of the first metatarsal 412. The knob 414 of the screw 404 is then threaded clockwise to compress the IM angle around a trial device (shown in FIG. 5). The rotational jig 200 can then be used to further compress the joint around the trial device to visualize correction. The optimally sized implant 102 then replaces the trial device in the joint space. The rotational jig 200 is then used to compress the first metatarsal 412 to the implant 102.

As shown in FIG. 5, a trial device 500 and inserter 502 are used to find the optimally sized implant 102. Specifically, a plurality of trial devices 500 of differing sizes can be inserted into the joint space, to find the optimally sized implant 102 needed. Once found, the optimally sized implant 102 replaces the trial device 500 in the joint space and is inserted using the inserter shaft.

FIGS. 6A-B illustrate multiple views of the graft packing block 600. Specifically, the graft packing block 600 is provided that fits the contour of the wedge implant 102 and is used to aid in the packing of bone graft within the opening 108 of the wedge implant 102. In use, the wedge implant body 104 can be placed in the graft packing block 600, and then filled with graft material as desired by the user.

FIGS. 7A-D illustrate multiple views of the wedge screw sleeve 700 utilized to insert the temporary fixation screws 702. The wedge screw sleeve 700 comprises a shaft 704 with corresponding angled drill guide towers 706. The shaft 704 of the wedge screw sleeve 700 is cannulated, allowing the wedge screw sleeve 700 to slide over an inserter device 708. Specifically, the wedge screw sleeve 700 is slid over the inserter 708 and the drill guides 706 are aligned with the insertion features 106 on the wedge implant 102. Once in place, a threaded knob 710 is slid over the inserter shaft 708 to lock the wedge screw sleeve 700 to the shaft 708 and ensure the sleeve 700 is fully seated in the insertion feature 112 of the wedge implant 102. Drilling and inserting of the temporary fixation screws 702 can then be done through the drill guide towers 706 attached to the wedge screw sleeve 700. Furthermore, the wedge screw sleeve 700 can also act as a depth gauge for the screws 702 (as shown in FIG. 7D), such that a user knows how far down a screw 702 has been inserted and/or how deep to drill to insert a screw 702.

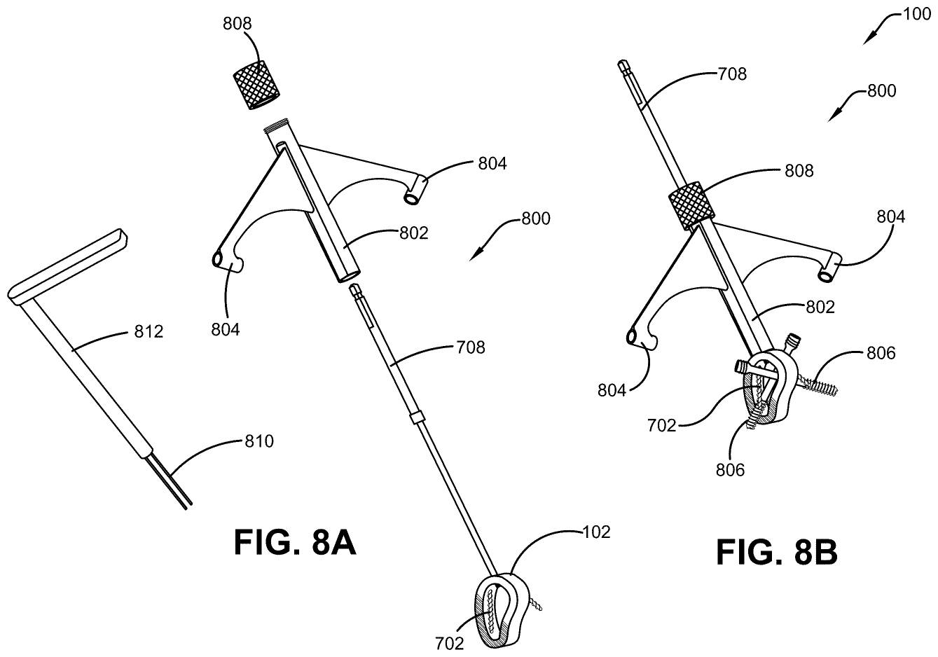

FIGS. 8A-B illustrate multiple views of the interfragmentary screw sleeve 800 used to target the space between the wedge screws 702 and provide compression through the wedge implant 102 and between the first metatarsal 412 and medial cuneiform. The interfragmentary screw sleeve 800 comprises a shaft 802 with corresponding angled drill guide towers 804. The drill guide towers 804 can be positioned at any suitable angle as is known in the art, as long as the interfragmentary screws 806 do not contact the wedge screws 702. The shaft 802 of the interfragmentary screw sleeve 800 is cannulated, allowing the interfragmentary screw sleeve 800 to slide over the inserter device 708. Specifically, the knob 710 from the wedge screw sleeve 700 is un-threaded and removed, and the wedge screw sleeve 700 is then removed, while keeping the inserter 708 threaded into the wedge implant 102. The interfragmentary screw sleeve 800 is then slid over the inserter 708 and the drill guides 804 are positioned. Once in place, a threaded knob 808 is slid over the inserter shaft 708 to lock the interfragmentary screw sleeve 800 to the shaft 708 and ensure the sleeve 800 is fully seated in the insertion feature 112 of the wedge implant 102. K-wires 810 are then utilized to ensure the correct trajectory of the two opposing interfragmentary screws 806. The K-wires 810 comprise a K-wire sleeve 812 which aids in insertion of the K-wire 810. Once the K-wire 810 is in position, the K-wire sleeve 812 is removed and a cannulated interfragmentary screw 806 is slid down the K-wire 810 and threaded into the bone. Then, the K-wire 810 is removed. Once the interfragmentary screws 806 are placed, the interfragmentary screw sleeve 800 and inserter 708 are removed, and the patient is closed.

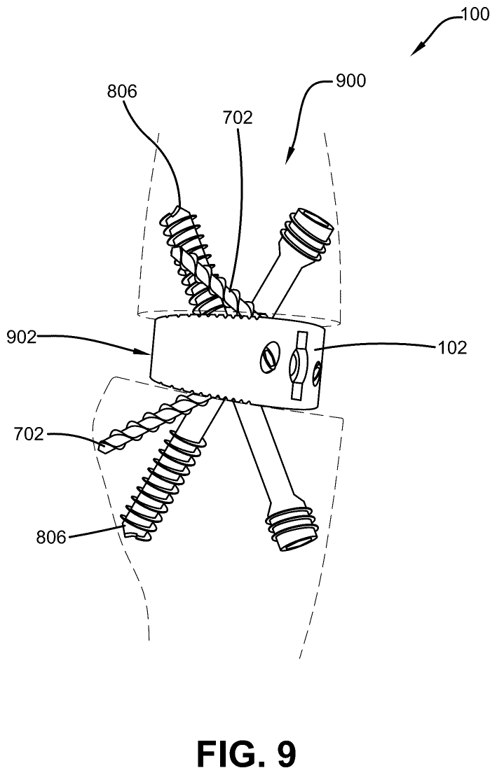

As shown in FIG. 9, the final configuration 900 discloses the wedge implant 102 restoring length to the first ray, with a set of temporary fixation screws 702 and a set of interfragmentary screws 806 securing the wedge implant 102 within the joint space 902.

A surgical method for implanting the wedge implant 102 into a joint space 902, will now be described. The method utilizes some of the devices, instruments, features, aspects, components and the like described above, and therefore reference will be made to the above described embodiments, such as the illustrated embodiments presented in the figures and discussed above. However, such references are made for exemplary purposes only and are not intended to limit the surgical method beyond the specifically recited steps. Further, the surgical method may be discussed under the umbrella of particular bones, but such an application is not intended to be limiting and the method described herein may be used or conducted with bone or other tissue not specifically discussed herein without departing from the spirit and scope of the surgical method.

Create a dorsomedial incision over the 1^{st} TMT joint to expose the medial aspect of the joint and the base of the metatarsal. The incision should be approximately the length of the cut guide which is just under 3.5 cm long. Retract soft tissue as necessary to aid in ease of cut guide placement. The incision can be extended over the medial metatarsal to expose the MTP joint and preform a capsular and soft tissue release.

Remove cartilage and prepare the 1^{st} TMT joint for fusion. Utilize a slim osteotome to mobilize the joint and release any plantar soft tissue attachments. Ensure the metatarsal has good mobility to enable rotation by using fluoroscopy to assess the distal metatarsal and sesamoids.

For rotational correction, attach the rotational jig to the medial cuneiform using a Steinmann pin. Place another Steinmann pin through the slotted arch of the rotational jig into the medial aspect of the first metatarsal. Use this pin as a joystick and glide the pin up the center of the arch and position it in the next highest slot to perform coronal correction and rotate the sesamoids plantar.

Further, parallel cuts are then needed. Place the paddle of the cut guide into the first TMT joint and firmly press down on the center of the cut guide to seat it close to the bone. Thread the joystick into the cut guide and use it to move the guide more dorsally or medially. Place k-wires in the preferred distal and proximal holes in the cut guide to fixate the guide in its optimal position. Ensure both wires are bi-cortical but not into the 2^{nd} metatarsal. Use a sagittal saw to make equal, parallel cuts through the designated slots on the medial cuneiform and first metatarsal. After the cuts are made, remove the cut guide and k-wires, and subsequently remove the remaining bone slivers. For a more severe bunion, the cut guide will be positioned slightly more medial than dorsal. Care should be taken to not cut into the second metatarsal base.

While maintaining the correction with the rotational jig, use the provided trial wedges to determine the appropriate size implant. Start with smaller trials and increase the trial footprint, thickness, and biplanar correction as desired. Make a stab incision lateral the head of the second metatarsal. Place the lateral hook of the IM angle reducer around the neck of the 2^{nd} metatarsal. Place the medial hook over the skin onto the head of the 1^{st} metatarsal. Turn the knob clockwise until it is snug and compress the bone to the trial. Use the knob on the rotational jig to further compress around the trial if necessary. Use fluoroscopy to confirm positioning. Once satisfied with the correction, distract one or two turns using the rotational jig to prepare for wedge insertion.

Use the inserter shaft to insert the selected implant into the joint space until the implant is sub-flush and/or the inserter reaches the stop. Fluoroscopy can be used to confirm the desired correction of deformity is achieved once the implant is in place. Once the implant is placed in the desired location, slide the Wedge Screw Sleeve over the inserter shaft. Ensure the insertion features on the sleeve are aligned with those on the wedge and are fully seated into the wedge. Tighten the knob attached to the sleeve onto the inserter shaft to lock it in place. Further, a graft packing block and tamp are available to insert bone graft into the implant's graft window before insertion.

Keeping the wedge in position, drill through the drill guide towers and refer to the depth indicator lines to determine screw length. Then advance the appropriate screw until the screw inserter reaches the stop on the wedge tower. Repeat the same for the remaining screw. In case of loss in screw engagement, the wedge inserter towers may be removed to finish screw insertion. Screws should be inserted until they are just sub-flush of the implant. Over-insertion could cause loss of engagement between the screw and the wedge. Turn the inserter shaft counterclockwise to disengage from the wedge. If needed, further drive the wedge screws until they are sub-flush with the implant.

Once the wedge screws are placed, additional fixation is required, and the selection of such fixation is surgeon preference. The Lapidus system provides a drill guide for two opposing Interfrag screws going through the wedge system, but the use of any other ancillary fixation is permitted.

To use the Interfrag Screw Sleeve, remove the inserter knob from the inserter shaft by turning counterclockwise and subsequently remove the Wedge Screw Sleeve, while maintaining the position of the inserter shaft within the wedge. Slide right (R) or left (L) Interfrag Screw Sleeves onto the inserter shaft and thread the knob in the same manner as before to lock in place. Ensure the arm marked with a ‘D’ is facing distally, and confirm the sleeve is fully seated into the insertion feature of the wedge. Once the sleeve is set, drive k-wires through the k-wire guides of the sleeve. Use fluoroscopy to confirm trajectory of Interfrag screws. Remove k-wire guides and drill over the wire. Slide cannulated screws onto the wire and advance to desired depth. Once the Interfrag screws are placed, remove the Interfrag Screw Sleeve and Inserter shaft, and close the patient.

Certain terms are used throughout the following description and claims to refer to particular features or components. As one skilled in the art will appreciate, different users may refer to the same feature or component by different names. This document does not intend to distinguish between components or features that differ in name but not structure or function. As used herein “length restoring Lapidus implant system”, “Lapidus system”, and “system” are interchangeable and refer to the length restoring Lapidus implant system 100 of the present invention.

Notwithstanding the forgoing, the length restoring Lapidus implant system 100 of the present invention can be of any suitable size and configuration as is known in the art without affecting the overall concept of the invention, provided that it accomplishes the above stated objectives. One of ordinary skill in the art will appreciate that the length restoring Lapidus implant system 100 as shown in FIGS. 1-9 is for illustrative purposes only, and that many other sizes and shapes of the length restoring Lapidus implant system 100 are well within the scope of the present disclosure. Although the dimensions of the length restoring Lapidus implant system 100 are important design parameters for user convenience, the length restoring Lapidus implant system 100 may be of any size that ensures optimal performance during use and/or that suits the user's needs and/or preferences.

Various modifications and additions can be made to the exemplary embodiments discussed without departing from the scope of the present invention. While the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

What has been described above includes examples of the claimed subject matter. It is, of course, not possible to describe every conceivable combination of components or methodologies for purposes of describing the claimed subject matter, but one of ordinary skill in the art may recognize that many further combinations and permutations of the claimed subject matter are possible. Accordingly, the claimed subject matter is intended to embrace all such alterations, modifications and variations that fall within the spirit and scope of the appended claims. Furthermore, to the extent that the term “includes” is used in either the detailed description or the claims, such term is intended to be inclusive in a manner similar to the term “comprising” as “comprising” is interpreted when employed as a transitional word in a claim.

What is claimed is:

1. A length restoring Lapidus implant system comprising:
a wedge implant; and
a plurality of instrumentation, wherein the length restoring Lapidus implant system maintains length and optimizes positioning of a first ray in both transverse and sagittal planes and allows for correction of axial sesamoids.

2. The length restoring Lapidus implant system of claim 1, wherein the plurality of instrumentation comprises a rotational jig, a parallel cut guide, and an interfragmentary screw sleeve, which allow for rotational correction, as well as provide compression.

3. The length restoring Lapidus implant system of claim 2, wherein the plurality of instrumentation further comprises an IM angle reducer, a trial wedge inserter, a graft packing block, and a wedge screw sleeve.

4. The length restoring Lapidus implant system of claim 3 wherein the wedge implant is made to a desired restoration length necessary for a user and has an outer anatomical profile that mimics anatomical considerations of bones it is being inserted between.

5. The length restoring Lapidus implant system of claim 4, wherein the outer anatomical profile of the wedge implant on a proximal and distal sides is cut to mimic shapes of medial cuneiform and metatarsal bones, which is a kidney shape.

6. The length restoring Lapidus implant system of claim 5, wherein the wedge implant is cut with a desired angle thereby creating a desired angular offset between the medial cuneiform and the metatarsal bones.

7. The length restoring Lapidus implant system of claim 6, wherein the desired angulation is used to correct valgus and plantar angulation of the metatarsal bones by tapering the wedge implant from a medial side to a lateral side and from a dorsal side to a plantar side.

8. The length restoring Lapidus implant system of claim 7, wherein the wedge implant comprises a body comprising at least two bores each of which are dimensioned to receive a bone screw, wherein the at least two bores can be designed for different angulation.

9. The length restoring Lapidus implant system of claim 8, wherein the wedge screw sleeve is utilized to insert at least one temporary fixation screw to secure the wedge implant in place and comprises a shaft with corresponding angled drill guide towers.

10. The length restoring Lapidus implant system of claim 9, wherein the shaft of the wedge screw sleeve is cannulated, allowing the wedge screw sleeve to slide over an inserter device.

11. The length restoring Lapidus implant system of claim 10, wherein insertion of the at least one temporary fixation screw is then done through the angled drill guide towers attached to the wedge screw sleeve, such that the at least one temporary fixation screw is inserted through the angled drill guide towers and through a bore and into surrounding bone to anchor the wedge implant in position.

12. The length restoring Lapidus implant system of claim 11, wherein the interfragmentary screw sleeve is used to target space between the at least one temporary fixation screw and provides compression through the wedge implant and between a first metatarsal and medial cuneiform.

13. The length restoring Lapidus implant system of claim 12, wherein the interfragmentary screw sleeve comprises a shaft with corresponding angled drill guide towers, such that the corresponding angled drill guide towers are positioned to not contact the at least one temporary fixation screw.

14. The length restoring Lapidus implant system of claim 13, wherein the shaft of the interfragmentary screw sleeve is cannulated, allowing the interfragmentary screw sleeve to slide over an inserter device.

15. A length restoring Lapidus implant system comprising:
a wedge implant comprising a body comprising at least two bores each of which are dimensioned to receive a bone screw at different angulations;
an IM angle reducer for lessening the IM angle;
a trial wedge inserter for inserting the trial wedges;
a graft packing block for inserting graft;
a wedge screw sleeve utilized to insert at least one temporary fixation screw and comprises a shaft with corresponding angled drill guide towers;
a rotational jig for rotating metatarsal;
a parallel cut guide for aligning cuts in joint; and
an interfragmentary screw sleeve utilized to target space between the at least one temporary fixation screw and provides compression through the wedge implant and between a first metatarsal and medial cuneiform;
wherein an outer anatomical profile of the wedge implant on a proximal and distal sides is cut to mimic shapes of medial cuneiform and metatarsal bones, which is a kidney shape;
wherein desired angulation of the wedge implant is used to correct valgus and plantar angulation of the metatarsal bones by tapering the wedge implant from a medial side to a lateral side and from a dorsal side to a plantar side;
wherein the wedge screw sleeve allows for insertion of the at least one temporary fixation screw to anchor the wedge implant in place;
wherein the interfragmentary screw sleeve allows for insertion of the interfrag screws to provide compression; and
further wherein the length restoring Lapidus implant system maintains length and optimizes positioning of a first ray in both transverse and sagittal planes and allows for correction of axial sesamoids.

16. The length restoring Lapidus implant system of claim 15, wherein the shaft of the wedge screw sleeve and the interfragmentary screw sleeve is cannulated.

17. The length restoring Lapidus implant system of claim 15, wherein insertion of the at least one temporary fixation screw is then done through the angled drill guide towers attached to the wedge screw sleeve, such that the at least one temporary fixation screw is inserted through the angled drill guide towers and through a bore and into surrounding bone to anchor the wedge implant in position.

18. The length restoring Lapidus implant system of claim 15, wherein the interfragmentary screw sleeve comprises a shaft with corresponding angled drill guide towers, such that the corresponding angled drill guide towers are positioned to not contact the at least one temporary fixation screw.

19. The length restoring Lapidus implant system of claim 18, wherein interfrag screws are then inserted via the interfragmentary screw sleeve to provide compression through the wedge implant and between a first metatarsal and medial cuneiform.

20. A method of maintaining length and optimizing positioning of a first ray in both transverse and sagittal planes, the method comprising the following steps:
making parallel cuts via placing a paddle of a cut guide into a first TMT joint and firmly pressing down to seat it close to bone;
after the cuts are made, removing the cut guide;
using an inserter shaft to insert a wedge implant into joint space;
once the wedge implant is in place, sliding a wedge screw sleeve over the inserter shaft;
keeping the wedge implant in position via drilling through drill guide towers, then advancing an appropriate screw;
once the wedge screws are placed, inserting a drill guide for two opposing interfrag screws;
sliding right or left interfrag screw sleeves onto an inserter shaft;
placing interfrag screws through the interfrag screw sleeves; and
removing the interfrag screw sleeves, after the interfrag screws are in place.
